(19) United States
(12) Patent Application Publication
(10) Pub. No.: US 2016/0038006 A1
(43) Pub. Date: Feb. 11, 2016

(54) ENDOSCOPE

(71) Applicant: FUJIKURA LTD., Tokyo (JP)

(72) Inventors: Kenichi NAKATATE, Sakura-shi (JP); Hitoe IIKURA, Sakura-shi (JP)

(73) Assignee: FUJIKURA LTD., Tokyo (JP)

(21) Appl. No.: 14/776,241

(22) PCT Filed: Oct. 21, 2013
(86) PCT No.: PCT/JP2013/078457

§ 371 Date: Sep. 14, 2015

(30) Foreign Application Priority Data
Mar. 25, 2013 (JP) 2013-062245

(51) Int. Cl. A61B 1/00; A61B 1/06; A61B 1/05

(52) U.S. Cl. CPC A61B 1/00096; A61B 1/051; A61B 1/0676

(57) ABSTRACT

An endoscope which is capable of backward viewing and the structure of whose tip section is reduced in size. The endoscope includes: an insertion unit; a tip section; and a connecting part. The insertion unit is inserted into a subject. The tip section includes a first illumination optical system for illuminating the inside of the subject, a first imaging device that captures an image of the interior of the subject, and a first imaging optical system having a first objective lens provided in front of the first imaging device. The connecting part connects the tip section to the insertion unit. The first imaging device is placed so that an imaging surface thereof faces the insertion unit.

### TECHNICAL FIELD

The invention relates to an endoscope.

### BACKGROUND ART

An endoscope is a medical device used for observing the interior of a subject. As solid-state imaging sensors (CCD sensors, CMOS sensors etc.) become more compact and have higher performance, endoscopes which have such solid-state imaging sensors at the ends of their insertion units (so-called electronic endoscopes) become common.

An electronic endoscope includes a flexible long insertion unit, for example. At a tip end of the insertion unit, an objective lens, a solid-state imaging sensor and the like are placed. Such an electronic endoscope is inserted into a subject and is capable of observing an area in front of the insertion unit.

When an electronic endoscope is however used to observe an pleated organ (e.g. small intestine), projecting lesion (e.g. polyps) or the like, observing the area in front of the insertion unit may be insufficient to eliminate the blind spots (e.g. back of pleats or polyps).

In order to solve this problem, an endoscope which can switch forward viewing and backward viewing is provided (see Patent Literatures 1 and 2). In the endoscope described in Patent Literature 1, the switching between forward viewing and backward viewing are achieved by switching a mirror 20a.

### CITATION LIST

### Patent Literature

[Patent Literature 1] Japanese Unexamined Patent Application Publication No. 2011-160998.

[Patent Literature 2] Japanese Unexamined Patent Application Publication No. 2012-40078

### SUMMARY OF INVENTION

### Technical Problem

But, the configurations according to Patent Literatures 1 and 2 needs a member dedicated to backward viewing (e.g. the mirror 20a of Patent Literature 1). Accordingly, there is a problem that the tip structures of the insertion units increase in size.

The invention is made to solve the foregoing problem, and an aspect thereof is to provide an endoscope which is capable of backward viewing and the structure of whose tip section is reduced in size.

### Solution to Problem

A primary aspect of the invention is an endoscope including a insertion unit, a tip section and a connecting part. The insertion unit is inserted into a subject. The tip section includes a first illumination optical system for illuminating the inside of the subject, a first imaging device that captures an image of the interior of the subject, and a first imaging optical system having a first objective lens provided in front of the first imaging device. The connecting part connects the tip section to the insertion unit. The first imaging device is placed so that an imaging surface thereof faces the insertion unit. Other features of this invention will become apparent from the description in this specification and the attached drawings.

### Advantageous Effects of Invention

An endoscope according to the invention is capable of backward viewing, and the structure of its tip section is reduced in size.

### BRIEF DESCRIPTION OF DRAWINGS

FIG. 1 is a diagram showing an endoscope according to the first embodiment.

FIG. 2 is a diagram showing the endoscope according to the first embodiment.

FIG. 3 is a diagram showing the endoscope according to the first embodiment.

FIG. 4 is a diagram illustrating additional details of the endoscope according to the first embodiment.

FIG. 5 is a diagram showing an endoscope according to the second embodiment.

FIG. 6 is a diagram illustrating additional details of the endoscope according to the second embodiment.

FIG. 7 is a diagram showing an endoscope according to the third embodiment.

FIG. 8 is a diagram showing the endoscope according to the third embodiment.

### DESCRIPTION OF EMBODIMENTS

(Overview)

With the description and the accompanied drawings, at least the following matters will be apparent.

An endoscope, including: a insertion unit that is inserted into a subject; a tip section including a first illumination optical system for illuminating the inside of the subject, a first imaging device that captures an image of the interior of the subject and that is placed so that an imaging surface thereof faces the insertion unit, and a first imaging optical system having a first objective lens provided in front of the first imaging device; and a connecting part that connects the tip section to the insertion unit.

Such an endoscope is capable of backward viewing, and the structure of its tip section is reduced in size.

An endoscope will be described wherein the tip section is inclined to the axial direction of the insertion unit.

With such an endoscope, a wide field of view can be achieved.

An endoscope will be described wherein an angle at which the tip section is inclined to the axial direction of the insertion unit is an angle at which the connecting part and the insertion unit do not come into an angle of view of the first objective lens.

With such an endoscope, a wide field of view can be achieved.

An endoscope will be described wherein the connecting part is made of a material containing shape memory alloy.

With such an endoscope, the connecting part can restore its original shape even when the connecting part is deformed by exerting a force on the tip section, for example.

An endoscope will be described wherein a diameter of the tip section is smaller than a diameter of the insertion unit.

Such an endoscope is easy to insert into a subject and the like.

An endoscope will be described wherein the first imaging device is a CMOS sensor.

With such an endoscope, the tip section can be further reduced in size.

An endoscope will be described wherein the tip section includes a second illumination optical system for illuminating the inside of a subject, a second imaging device that captures an image of the interior of the subject and that is placed so that an imaging surface thereof faces the opposite side of an imaging surface of the first imaging device, and a second imaging optical system having a second objective lens provided in front of the second imaging device.

Such an endoscope is capable of forward viewing as well as backward viewing.

### First Embodiment

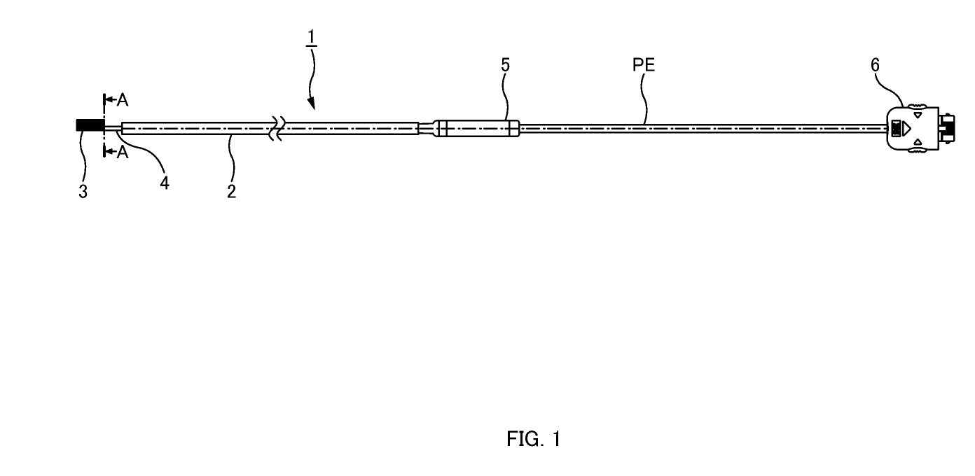

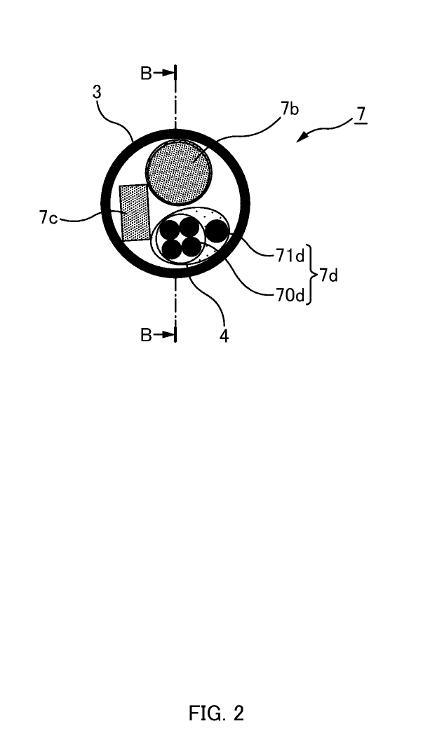

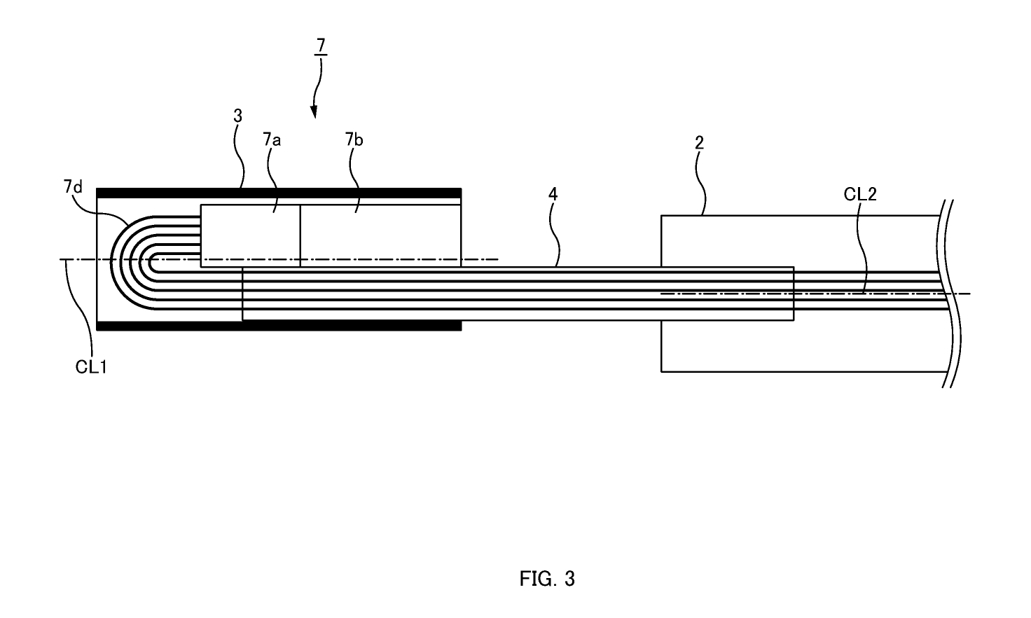

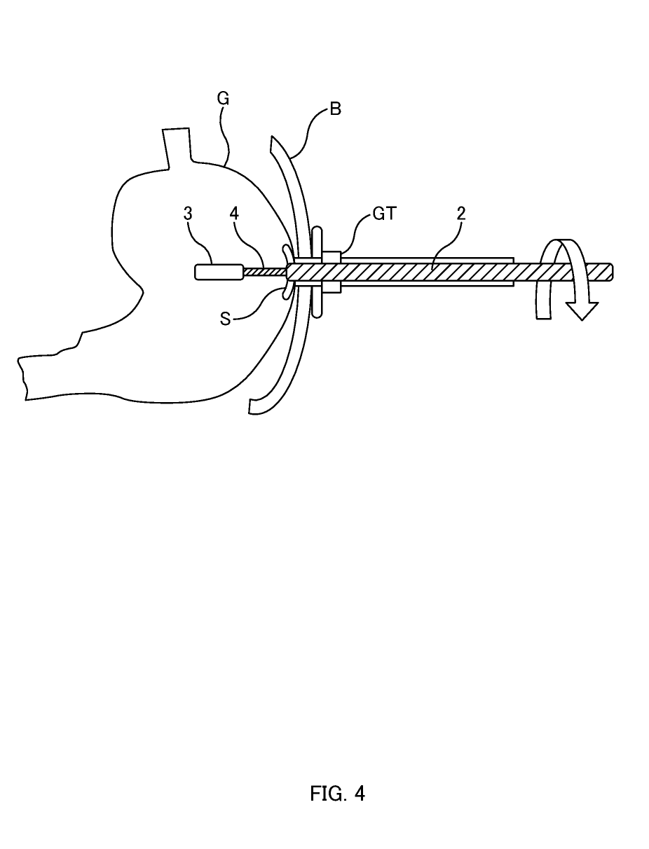

With reference to FIGS. 1 to 4, the configuration of an endoscope according to the first embodiment 1 will be described. FIG. 1 is a diagram showing an overall appearance of an endoscope 1. FIG. 2 is a cross-sectional view taken along line A-A in FIG. 1. FIG. 3 is a cross-sectional view taken along line B-B in FIG. 2. FIG. 4 is a schematic diagram (cross-sectional view) showing a usage example of the endoscope 1 according to the present embodiment.

<Configuration>

The endoscope 1 is a medical device used to observe the interior of a subject. The endoscope 1 includes: an insertion unit 2; a tip section 3; a connecting part 4; a handle 5; and a connector 6 (see FIG. 1). The endoscope 1 according to the present embodiment includes at least an insertion unit 2, a tip section 3 and a connecting part 4.

The insertion unit 2 is an elongated, cylindrical member to be inserted into a subject. The insertion unit 2 is flexible, and is formed by, for example, coating a cylindrical metal member with resin (polyurethane, polyethylene, fluoropolymers, etc). The diameter of the insertion unit 2 is 3.0 mm, for example.

The tip section 3 is a tip end of the endoscope 1 and is a cylindrical hard member. The tip section 3 is made of, for example, stainless steel (e.g. SUS304). The diameter of the tip section 3 is 2.7 mm, for example. In the present embodiment, the tip section 3 has a diameter smaller than that of the insertion unit 2. This makes it easier to insert the endoscope 1 because the tip section 3 becomes less likely to be hooked when the endoscope 1 is inserted into a subject.

Inside the tip section 3, a imaging optical system 7 is placed (see FIGS. 2 and 3). The imaging optical system 7 includes: an imaging device 7a; an objective lens 7b; an LED light source 7c serving as an illumination optical system; and a cable section 7d (see FIGS. 2 and 3). The imaging optical system 7 is fixed inside the tip section 3 using resin adhesive and the like. The imaging optical system 7 according to the present embodiment includes at least an imaging device 7a, an objective lens 7b and the illumination optical system (an LED light source 7c). The imaging optical system 7 according to the present embodiment is an example of the “first imaging optical system”.

The imaging device 7a is a device which captures images of the interior of a subject. Within the tip section 3, the imaging device 7a according to the present embodiment is placed so that its imaging surface faces the insertion unit 2 (see FIG. 3). As the imaging device 7a, CMOS sensors or CCD sensors may be used, for example. CMOS sensors are more suitable for size reduction of the tip section 3, compared to CCD sensors. The imaging device 7a according to the present embodiment is an example of the “first imaging device”.

The objective lens 7b is provided in front of the imaging surface of the imaging device 7a inside the tip section 3. In the present embodiment, the objective lens 7b is placed so that one of its lens surfaces (the surface opposite to the lens surface facing the imaging surface) coincides with the back face of the tip section 3. The imaging device 7a captures images of the view behind the tip section 3 (so-called backward viewing) through the objective lens 7b. The objective lens 7b may be a GRIN lens, for example. Or, the objective lens 7b may be a lens group in which a plurality of lenses (glass, plastic, etc.) are combined. The angle of view of the objective lens 7b may be 95°-120°, for example. In the present embodiment, the imaging device 7a and the objective lens 7b are placed at eccentric positions with respect to the center of the cross section of the tip section 3 (see FIG. 3). The objective lens 7b according to the present embodiment is an example of the “first objective lens”.

The LED light source 7c is provided inside the tip section 3 and illuminates a subject. Inside the tip section 3, the LED light source 7c is placed so that a light-emitting surface is located near the objective lens 7b (that is, the LED light source 7c is placed so that its light-emitting surface faces the insertion unit 2). The LED light source 7c is supplied with driving power through the cable section 7d to illuminate a subject. Using the LED light source 7c as the illumination optical system can make the diameter of the insertion unit 2 smaller.

The illumination optical system is not limited to the LED light source 7c. A light-guide fiber may be used as the illumination optical system. The light-guide fiber guides light from a light source (not shown) into a subject. Inside the tip section 3, the light-guide fiber is placed so that its light-emitting surface faces the insertion unit 2. The root end of the light-guide fiber is inserted through the connecting part 4 and the insertion unit 2 and is connected to the light source (not shown) placed outside the subject. A plurality of the light-guide fibers may be provided. Use of the light-guide fibers increases light which illuminates the subject.

The cable section 7d includes a plurality of imaging-device cables 70d and LED cables 71d. The imaging-device cables 70d and the LED cables 71d may be super fine coaxial cables, for example. The imaging-device cables 70d are wires to transmit driving signals (and driving power) for driving the imaging device 7a, image signals (signals which are captured images converted into electrical signals) from the imaging device 7a, and the like. A tip end of each imaging-device cable 70d is connected to the imaging device 7a. The LED cables 71d are wires to transmit driving signals (and driving power) for driving the LED light source 7c. A tip end of each LED cable 71d is connected to the LED light source 7c. The root end of the cable section 7d (the imaging-device cable 70d and the LED cable 71d) is inserted through the connecting part 4 and the insertion unit 2, and is connected to a processor (not shown) via the connector 6. The processor (not shown) is a unit placed outside a subject. The processor (not shown) performs the function of processing image signals to form an image, and the function of supplying driving power to the imaging device 7a and the LED light source 7c. The cable section 7d and the imaging device 7a can be electrically connected by providing an FPC board and the like therebetween.

The connecting part 4 is a cylindrical member which connects the tip section 3 to the insertion unit 2. One end of the connecting part 4 is placed inside the tip section 3, and the other end is placed inside the insertion unit 2 The connecting part 4 is fixed, using adhesive etc, to the tip section 3 and the insertion unit 2. The connecting part 4 has a diameter smaller than those of the tip section 3 and the insertion unit 2. Inside the connecting part 4, the cable section 7d is inserted. In the present embodiment, the tip section 3, the insertion unit 2 and the connecting part 4 are placed straight (see FIG. 1). However, in order to ensure the imaging optical system 7 to have a field of view of an appreciable size, the tip section 3 is placed so that the axis CL1 (the axial direction) of the tip section 3 does not coincide with the axis CL2 (the axial direction) of the insertion unit 2 (see FIG. 3).

The connecting part 4 is made of polyimid or fluoropolymer, for example. Or, the connecting part 4 may be made of shape memory alloy. Specifically, the entire body of the connecting part 4 may be made of shape memory alloy, and the connecting part 4 may also be made of cylindrical resin into which wire-like shape memory alloy is inserted. In this case, if the connecting part 4 is deformed (if the tip section 3 is bent with respect to the insertion unit 2) by exerting a force on the tip section 3 and the like, the connecting part 4 can restore its original shape (e.g. the straight shape in FIG. 1).

The handle 5 is a part to be held when the endoscope 1 is operated for (inserted into or drawn out) a subject. A operator such as a physician holds the handle 5 in one hand, and pushes the insertion unit 2 by the other hand into a medical tube or a channel of the endoscope. Or, the operator rotates the endoscope 1 (the insertion unit 2) by twisting the handle 5.

The connector 6 is a component electrically connecting the processor (not shown) to the endoscope 1. The part of the cable section 7d from the handle 5 to the connector 6 is covered with polyethylene tube PE, for example (see FIG. 1).

<Usage Example of Endoscope 1>

The endoscope 1 having the above configuration can be used in various sites of a subject. At least the length of the insertion unit 2 is different depending on a site for which the endoscope 1 is used.

For example, there is a method for administering nutrients directly to the stomach of a patient whom oral intake of food is impossible (Percutaneous Endoscopic Gastrostomy: hereinafter referred to as “PEG”).

Specifically, PEG is a method for making an incision in the abdomen B (the stomach G) of a subject using an endoscope. A gastrostomy tube GT is inserted through the incision made in PEG and fixed. The gastrostomy tube GT is a hollow member having a stopper S at its tip section (see FIG. 4). The stopper S extends the inside of the stomach G and thus the gastrostomy tube GT is fixed to stomach wall. Thus, an operator such as a nurse can administer nutrients directly to the stomach G of a subject through the gastrostomy tube GT.

In order to fix the gastrostomy tube GT to the inside of the stomach G, the stopper S is required to extend successfully. It is impossible to supply nutrients when the gastrostomy tube GT is closed, and a conventional endoscope has been inserted orally or nasally to examine this state. On the other hand, the small tip section 3 and the entire body of the endoscope 1 according to the present embodiment has a small diameter. Accordingly, the endoscope 1 can be inserted into a subject through the gastrostomy tube GT. That is, the subject's burden is reduced compared to cases of inserting an endoscope orally or nasally.

If the endoscope 1 is inserted into the gastrostomy tube GT, the stopper S is located behind the tip section 3 (see FIG. 4). The endoscope 1 according to the present embodiment includes the imaging optical system 7 capable of backward viewing. Accordingly, it is possible to examine the state of the stopper S within the field of view F of the imaging optical system 7 (the objective lens 7b) (see FIG. 4), for example, by rotating the tip section 3 through the insertion unit 2 (the arrow in FIG. 4 indicates the rotational direction of the endoscope 1).

The endoscope 1 may be applied to various medical tubes (endotracheal tubes, ileus tubes, etc) used for a subject as well as to the gastrostomy tube GT. And, the endoscope 1 can be inserted into a subject through a channel of a common endoscope (e.g. digestive tract endoscope). That is, the endoscope 1 according to the present embodiment can serve as an auxiliary scope for a common endoscope. In addition to indirect insertion of the endoscope 1 into a subject through a medical tube etc, it goes without saying that the endoscope 1 can be inserted directly.

Thus, the endoscope 1 according to the present embodiment is capable of backward viewing. Accordingly, the endoscope 1 may be employed in different use from a common endoscope (or an auxiliary use in observation with a common endoscope). The imaging device 7a (the imaging surface of the imaging device 7a) faces backward (toward the insertion unit 2) and is capable of backward viewing. Accordingly, since any special structure for backward viewing (e.g. a mirror) is not necessary, the tip section 3 of the endoscope 1 can be reduced in size and production costs thereof can be reduced.

### Second Embodiment

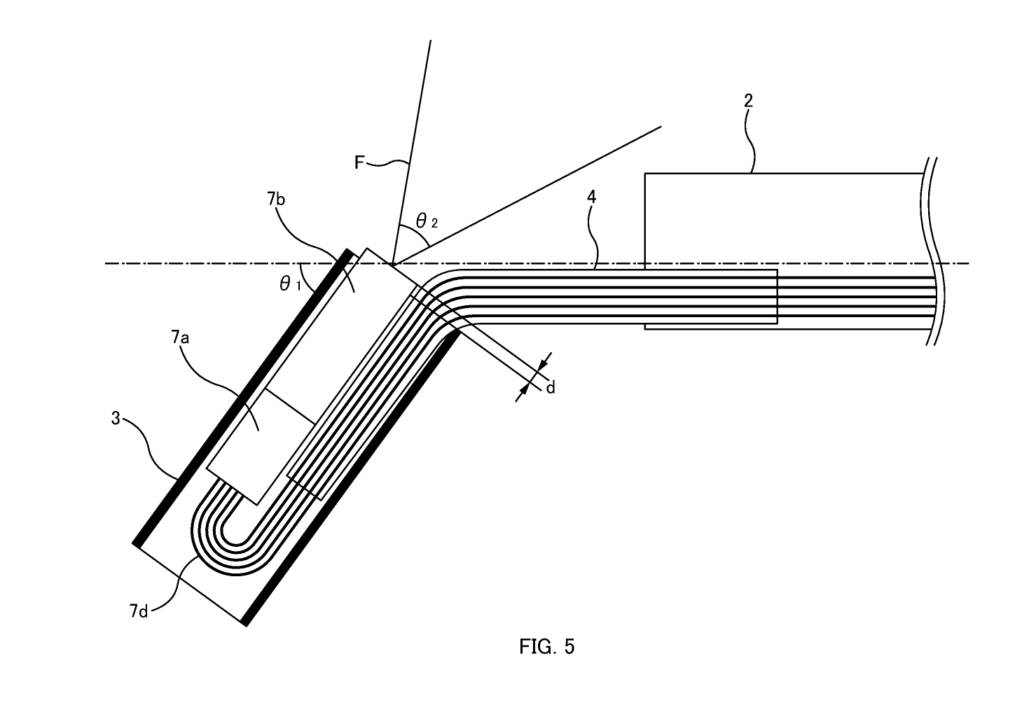

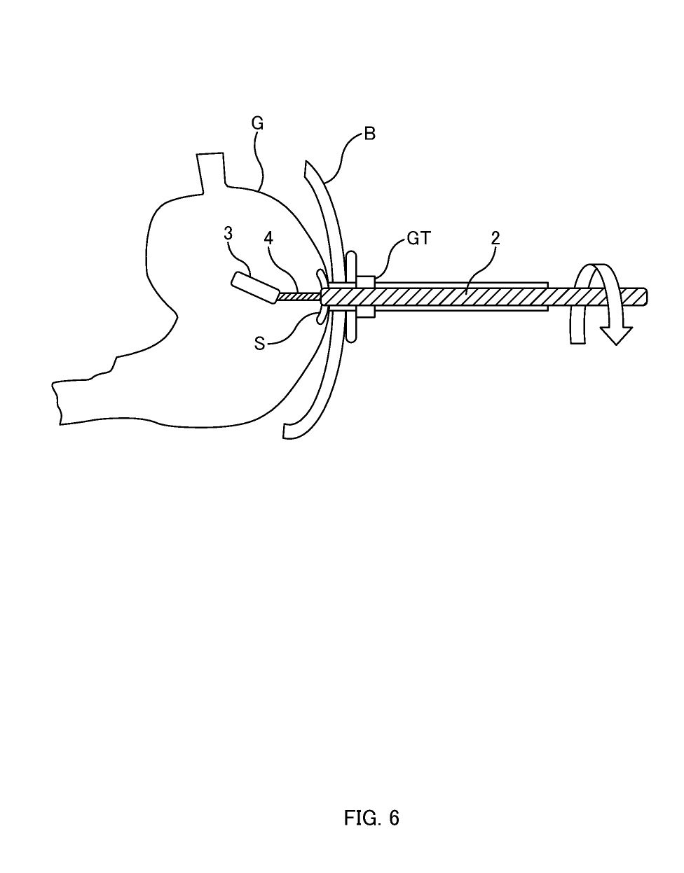

With reference to FIGS. 5 and 6, the configuration of an endoscope 1 according to the second embodiment will be described. In the present embodiment, an example in which the tip section 3 is inclined to the axial direction of the insertion unit 2 will be described. FIG. 5 is a cross-sectional view of the endoscope 1 according to the present embodiment (a part of the insertion unit 2, a tip section 3 and the connecting part 4). FIG. 6 is a schematic diagram (cross-sectional view) showing a usage example of the endoscope 1 according to the present embodiment. The detailed description of the same structure as the first embodiment is omitted.

<Configuration>

The tip section 3 in the present embodiment is inclined to the axial direction of the insertion unit 2 at a predetermined angle (an inclination angle θ1). One of the lens surface (the surface opposite to the lens surface facing the imaging surface) of the objective lens 7b protrudes beyond the back face of the tip section 3 by a distance d.

The inclination angle θ1 is an angle within which the insertion unit 2 and the connecting part 4 do not come into the field of view F of the objective lens 7b. The inclination angle θ1 is determined, if the insertion unit 2 and the connecting part 4 each have a uniform diameter and if the distance from the insertion unit 2 to the tip section 3 is constant, according to the relation between the angle of view θ2 of the objective lens 7b and the distance d from the back face of the tip section 3 to the one lens surface of the objective lens 7b.

For example, if the objective lens 7b having a wide angle of view θ2 is used, when the objective lens 7b is fixed to the tip section 3, the distance d is set long. This makes it possible for the insertion unit 2 and the connecting part 4 not to come into the field of view F (angle of view θ2).

The connecting part 4 is partially bent according to the inclination of the tip section 3. If the connecting part 4 is made of a material such as shape memory alloy, the connecting part 4 can restore its original shape (the state at inclination angle θ1) even when the connecting part 4 is deformed by exerting a force on the tip section 3. The objective lens 7b is placed on a side different from the side toward which the connecting part 4 is bent (the outer side of the bent connecting part 4).

<Usage Example of Endoscope 1>

FIG. 6 shows an example in which the endoscope 1 according to the present embodiment is used for a gastrostomy tube GT.

As in the first embodiment, the small tip section 3 and the entire body of the endoscope 1 according to the present embodiment has a small diameter. Accordingly, the endoscope 1 can be inserted into a subject through the gastrostomy tube GT. That is, the subject's burden is reduced compared to cases of inserting an endoscope orally or nasally.

Also as in the first embodiment, the endoscope 1 according to the present embodiment includes the imaging optical system 7 capable of backward viewing. Accordingly, it is possible to examine the state of stopper S within the field of view F of the imaging optical system 7 (the objective lens 7b) (see FIG. 6), for example, by rotating the tip section 3 through the insertion unit 2 (the arrow in FIG. 6 indicates the rotational direction of the endoscope 1).

In addition, in the endoscope 1 according to the present embodiment, the tip section 3 is inclined to the axial direction of the insertion unit 2. Accordingly, when examining the state of the stopper S by using the endoscope 1, the insertion unit 2 and the connecting part 4 do not come into the field of view F of the objective lens 7b.

Thus, in the endoscope 1 according to the present embodiment, since the insertion unit 2 and the connecting part 4 do not come into the field of view F of the objective lens 7b, a wide field of view can be achieved. Accordingly, observation with the endoscope 1 becomes more efficient.

### Third Embodiment

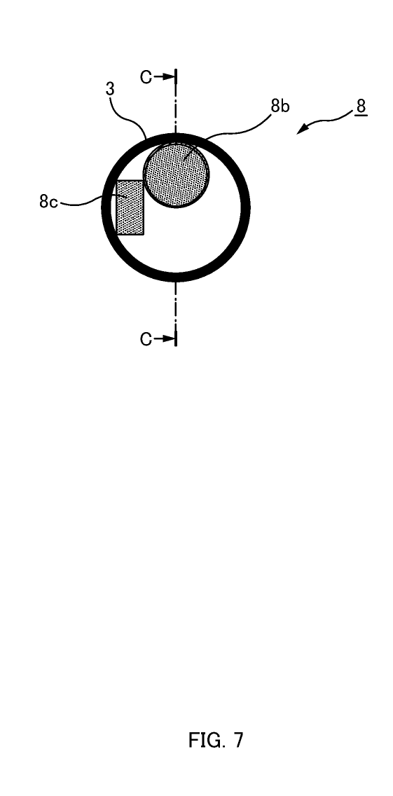

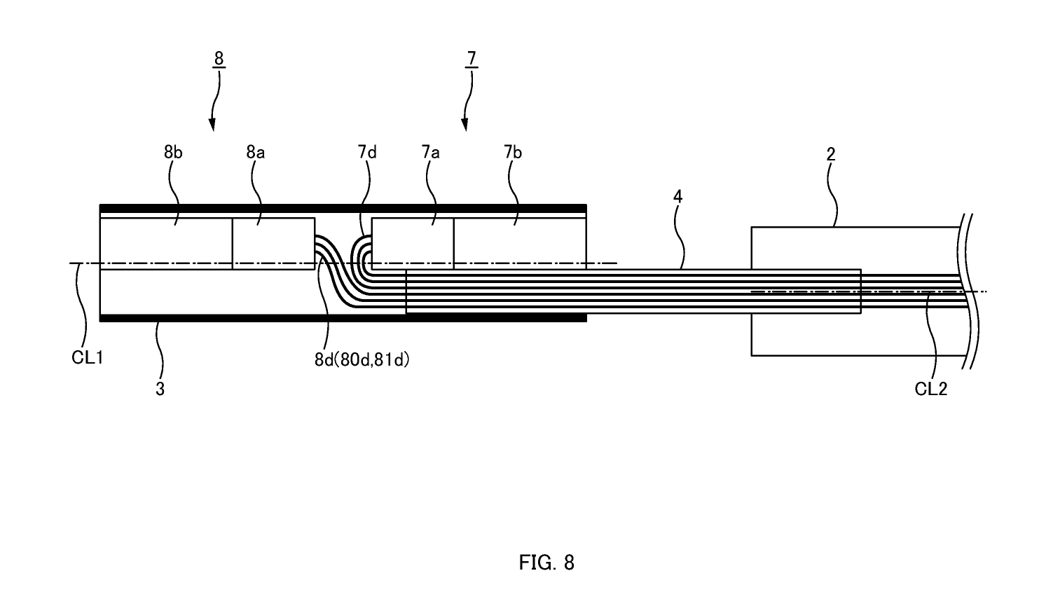

With reference to FIGS. 7 and 8, the configuration of an endoscope 1 according to the third embodiment will be described. In the present embodiment, an example in which a imaging optical system 8 for forward viewing (viewing the opposite side of the imaging optical system 7) is provided in addition to the imaging optical system 7 for backward viewing will be described. FIG. 7 shows the front face of the tip section 3 of the endoscope 1 according to the present embodiment. FIG. 8 is a cross-sectional view taken along line C-C in FIG. 7. The detailed description of the same structure as the first embodiment and the second embodiment is omitted. It is possible to appropriately combine the configurations of any of the first to third embodiments.

<Configuration>

Inside the tip section 3 according to the present embodiment, the imaging optical system 8 is placed as well as the imaging optical system 7. The imaging optical system 8 includes: an imaging device 8a; an objective lens 8b; an LED light source 8c serving as an illumination optical system; and a cable section 8d (see FIGS. 7 and 8). The imaging optical system 8 is fixed inside the tip section 3 using resin adhesive and the like. The imaging optical system 8 according to the present embodiment includes at least an imaging device 8a, an objective lens 8b and the illumination optical system (LED light source 8c). The imaging optical system 8 according to the present embodiment is an example of the “second imaging optical system”.

Inside the tip section 3, the imaging device 8a is placed so that its imaging surface faces the opposite side of the imaging surface of the imaging device 7a (see FIG. 8). In other words, the imaging surface of the imaging device 8a is placed so as to face the opposite side of the insertion unit 2 (the tip side of the tip section 3). The imaging device 8a according to the present embodiment is an example of the “second imaging device”.

The objective lens 8b is provided in front of the imaging surface of the imaging device 8a inside the tip section 3. The objective lens 8b is placed so that one of its lens surfaces (the surface opposite to the lens surface facing the imaging surface) coincides with the front face of the tip section 3. The imaging device 8a captures images of the view in front of the tip section 3 (forward viewing) through the objective lens 8b. In the present embodiment, the imaging device 8a and the objective lens 8b are placed at eccentric positions with respect to the center of the cross section of the tip section 3 (see FIG. 8). The objective lens 8b according to the present embodiment is an example of the “second objective lens”.

The LED light source 8c is placed inside the tip section 3 so that a light-emitting surface is located near the objective lens 8b (that is, the LED light source 8c is placed so that its light-emitting surface faces the opposite side of the insertion unit 2). The LED light source 8c is supplied with driving power through the cable section 8d to illuminate a subject.

The cable section 8d includes a plurality of imaging-device cables 80d and LED cables 81d, both of which are for the imaging device 8a and the LED light source 8c. The imaging-device cables 80d are wires to transmit driving signals (and driving power) for driving the imaging device 8a, image signals (signals which are captured images converted into electrical signals) from the imaging device 8a, and the like. A tip end of each imaging-device cable 80d is connected to the imaging device 8a. The LED cables 81d are wires to transmit driving signals (and driving power) for driving the LED light source 8c. A tip end of each LED cable 81d is connected to the LED light source 8c. The root end of the cable section 8d together with the cable section 7d is inserted through the connecting part 4 and the insertion unit 2, and is connected to a processor (not shown) via the connector 6.

The imaging optical system 7 and the imaging optical system 8 may have different configurations from each other. For example, the imaging device 7a may be a CMOS sensor, and the imaging device 8a may be a CCD sensor. Or, the objective lens 7b and the objective lens 8b may be lenses which have different angles of view θ2 from each other.

The endoscope 1 according to the present embodiment is capable of forward viewing as well as backward viewing. Accordingly, when the endoscope 1 is inserted into a subject, for example, through a gastrostomy tube GT, an operator can insert it while examining the front state in the insertion direction (e.g. whether the gastrostomy tube is closed or not). Or, through a channel of the endoscope, it is possible to observe a site (such as bile duct or small intestine) which cannot be observed with a common digestive-tract endoscope. Thus, the endoscope 1 according to the present embodiment can be applied for various use of observation.

### REFERENCE SIGNS LIST

1 endoscope
2 insertion unit
3 tip section
4 connecting part
5 handle
6 connector
7 imaging optical system
7a imaging device
7b objective lens
7c LED light source
7d cable section

1-7. (canceled)

8. An endoscope, comprising:
an insertion unit that is inserted into a subject;
a tip section including
a first illumination optical system for illuminating an inside of the subject,
a first imaging device
that captures an image of an interior of the subject and
that is placed so that an imaging surface thereof faces the insertion unit, and
a first imaging optical system having a first objective lens provided in front of the first imaging device; and
a connecting part that connects the tip section to the insertion unit.

9. An endoscope according to claim 8, wherein
the tip section is inclined to an axial direction of the insertion unit.

10. A endoscope according to claim 9, wherein
an angle at which the tip section is inclined to the axial direction of the insertion unit is an angle at which the connecting part and the insertion unit do not come into an angle of view of the first objective lens.

11. An endoscope according to claim 8, wherein
the connecting part is made of a material containing shape memory alloy.

12. An endoscope according to claim 8, wherein
a diameter of the tip section is smaller than a diameter of the insertion unit.

13. An endoscope according to claim 8, wherein
the first imaging device is a CMOS sensor.

14. An endoscope according to claim 8, wherein
the tip section includes
a second illumination optical system for illuminating an inside of a subject,
a second imaging device
that captures an image of an interior of the subject and
that is placed so that an imaging surface thereof faces an opposite side of an imaging surface of the first imaging device, and
a second imaging optical system having a second objective lens provided in front of the second imaging device.
